(19) United States
(12) Patent Application Publication
(10) Pub. No.: US 2020/0049697 A1
(43) Pub. Date: Feb. 13, 2020

(54) ENZYME-LINKED ASSAY FOR MEASUREMENT OF PORPHOBILINOGEN (PBG)

(71) Applicant: Board of Regents, The University of Texas System, Austin, TX (US)

(72) Inventor: Karl E. Anderson, Galveston, TX (US)

(73) Assignee: Board of Regents, The University of Texas System, Austin, TX (US)

(21) Appl. No.: 16/533,561

(22) Filed: Aug. 6, 2019

Related U.S. Application Data
(60) Provisional application No. 62/715,679, filed on Aug. 7, 2018.

(51) Int. Cl. G01N 33/50; G01N 33/493

(52) U.S. Cl. CPC G01N 33/5014; C12Y 205/01061; G01N 33/493

(57) ABSTRACT

The present invention includes a method and kit for detecting porphobilinogen fluorometrically comprising: obtaining a sample suspected of having a porphobilinogen; catalyzing a reaction in the sample with a porphobilinogen deaminase to form a porphyrin; exposing the porphyrin to a light source having a 399 nm wavelength; detecting fluorometrically the amount of porphyrin in the sample; and correlating the amount of porphyrin in the sample to an amount of porphobilinogen in the sample, wherein the ratio of porphobilinogen to porphyrin is 4:1.

### CROSS-REFERENCE TO RELATED APPLICATIONS

This application claims priority to U.S. Provisional Application Ser. No. 62/715,679, filed Aug. 7, 2018, the entire contents of which are incorporated herein by reference.

### STATEMENT OF FEDERALLY FUNDED RESEARCH

None.

### TECHNICAL FIELD OF THE INVENTION

The present invention relates in general to the field of porphyrias, and more particularly, to a rapid, novel enzyme-linked assay for measurement of porphobilinogen (PBG).

### BACKGROUND OF THE INVENTION

Without limiting the scope of the invention, its background is described in connection with porphyrias.

Porphyrias are rare disorders that result from alterations in activities of the eight enzymes of the heme biosynthetic pathway. These deficiencies plus environmental factors cause accumulation of intermediates of this pathway. These appear in blood, are excreted in urine and feces, and are associated with symptoms reflecting effects on the nervous system that usually occur as acute attacks (the acute porphyrias) or cutaneous photosensitivity (cutaneous porphyrias). Acute attacks of porphyria can be severe and life threatening, and prompt diagnosis is important so specific and effective treatment can be administered. The most common acute porphyria is acute intermittent porphyria (AIP); others are hereditary coproporphyria (HCP), variegate porphyria (VP), and 6-aminolevulinic acid dehydratase porphyria (ADP, which is extremely rare—only 8 cases are documented).

The neurological manifestations of acute porphyrias are associated with substantial elevations in δ-aminolevulinic acid (ALA, an amino acid committed to heme synthesis), which is elevated in all of these disorders, and porphobilinogen (PBG, a pyrrole), which is elevated in AIP, HCP and VP. Substantial elevation of PBG does not occur in any other medical conditions, so measuring urine PBG is a highly specific and sensitive diagnostic tool for these acute porphyrias. Elevation of ALA is not as great as PBG in AIP, HCP and VP, and it is also less specific in being elevated in some conditions other than acute porphyrias.

Porphyrias can be challenging to diagnose because they are rare and their symptoms are nonspecific. Biochemical diagnostic testing is essential and need not be complex if only one first-line test is used for screening, with further testing only when a screening test is positive. The specific screening test used depends on the type of porphyria suspected. Porphyrias are categorized into three clinical types, which guide the choice of screening tests (ie, there is no “porphyrin screen” that tests for all porphyrias).

Detecting PBG elevation is needed for timely diagnosis of the three most common acute porphyrias. PBG is colorless, but reacts with Ehrlich's aldehyde (p-dimethylaminobenzaldehyde) to form a reddish chromophore that is the basis for qualitative testing for elevated levels, and for quantitative spectrophotometric determination. Most previous methods for assessing PBG levels have relied on formation of this PBG chromophore. However, other substances in urine, most notably urobilinogen (a product of bilirubin breakdown in the gut) also react with Ehrlich' s aldehyde. Therefore, separation methods or conditions that especially favor formation of the PBG chromophore have been devised, but none are widely used.

Prior methods for separating PBG from urobilinogen and other contaminants have included the following.

Watson-Schwartz test. Ehrlich reagent is added to urine, and if a reddish color is formed, the sample is extracted with chloroform and then butanol. Intense color indicates the presence of excess PBG if the color remains in the aqueous phase. This test is reliable in experienced hands, but with occasional use is subject to misinterpretation, and is not quantitative.

Hoesch test. A few drops of urine are added to a small volume of strongly acidic Ehrlich reagent. A strongly reddish color indicates a high PBG concentration, since formation of the PBG chromophore is more favored under strongly acidic conditions compared to that formed with urobilinogen. This test is simple and rapid, but less sensitive than the Watson-Schwartz test and is not quantitative.

Mauzerall-Granick method. This is the standard quantitative method for measuring ALA and PBG, which are separated from each other and from interfering substances using two piggyback small plastic ion exchange columns followed by addition of Ehrlich reagent to the eluate containing PBG. Separated ALA is derivatized with acetylacetone to form a pyrrole that also reacts with Ehrlich reagent to form a chromophore. This method is dependable and accurate, but is not rapid.

Trace PBG Kit. The kit was invented in Australia and distributed by Thermo Scientific. It is essentially a separation method for PBG similar to the Mauzerall-Granick method. The ion exchange resin is provided in a plastic syringe, which facilitates drawing up the sample, followed by rinsing to remove interfering substances and then elution of the separated PBG. Ehrlich reagent is then added. A color chart is provided to facilitate accurate estimation of the concentration of PBG in the original sample. This kit was very useful, but was recently taken off the market presumably due to low sales volume. This has resulted in worldwide lack of a suitable method that can be recommended for use for screening for acute porphyrias.

Mass spectrometry (MS). Large laboratories have adopted MS methods to measure ALA and PBG. MS has much greater sensitivity, and the normal (reference) ranges are much lower than other methods. However, sensitivity is not an issue in screening for acute porphyrias because marked elevations are expected at the time of symptoms. The clinical significance of small elevations in PBG as detected by MS, but not the Mauzerall-Granick method, are not clear, and such small elevations have often led to misdiagnoses of acute porphyrias. MS is also not suitable as a rapid screening method for acute porphyrias.

Thus, a need remains for a rapid and dependable method for detecting PBG elevation for timely diagnosis of the three most common acute porphyrias.

### SUMMARY OF THE INVENTION

In one embodiment, the present invention includes a method for detecting porphobilinogen fluorometrically comprising: obtaining a sample suspected of having a porphobilinogen elevation above a normal level; catalyzing a reaction in the sample with a porphobilinogen deaminase to form a porphyrin reaction sample; exposing the porphyrin reaction sample to a light source having a 399 nm wavelength; detecting fluorometrically the amount of porphyrin in the sample at 620 to 700 nm; and correlating the amount of porphyrin in the reaction sample to an amount of porphobilinogen in the sample, wherein the ratio of porphobilinogen to porphyrin is 4:1. In one aspect, the sample is a urine sample. In another aspect, the sample is from a subject suspected of having acute intermittent porphyria (AIP). In another aspect, the sample is from a subject suspected of having hereditary coproporphyria (HCP). In another aspect, the sample is from a subject suspected of having variegate porphyria (VP). In another aspect, the method further comprises the step of adding porphobilinogen deaminase (PBGD) to the sample and measuring an increase in uroporphyrin I. In another aspect, the method further comprises the step of measuring uroporphyrinogen III in the sample. In another aspect, the method further comprises the step of measuring PBG in a tissue sample by inhibiting uroporphyrinogen III synthase (UROS) activity. In another aspect, the porphobilinogen deaminase is a human porphobilinogen deaminase. In another aspect, the porphobilinogen deaminase is immobilized on a substrate. In another aspect, the method further comprises the step of measuring the amount of porphyrin in an unreacted sample, and subtracting the amount of porphyrin in an unreacted sample from the reacted sample. In another aspect, the method further comprises the step of treating the subject having elevated porphobilinogen with carbohydrate loading or hemin, RNA interference, or gene therapy.

In another embodiment, the present invention includes a method for detecting porphobilinogen in a urine sample fluorometrically comprising: obtaining a urine sample suspected of having a porphobilinogen elevation above a normal level; catalyzing a reaction of the urine sample with a porphobilinogen deaminase to form a porphyrin; exposing the porphyrin to a light source having a 399 nm wavelength; detecting fluorometrically the amount of porphyrin in the urine sample; and correlating the amount of porphyrin in the urine sample to an amount of porphobilinogen in the sample, wherein the ratio of porphobilinogen to porphyrin is 4:1. In one aspect, the sample is from a subject suspected of having acute intermittent porphyria (AIP). In another aspect, the sample is from a subject suspected of having hereditary coproporphyria (HCP). In another aspect, the sample is from a subject suspected of having variegate porphyria (VP). In another aspect, the method further comprises the step of adding porphobilinogen deaminase (PBGD) to the sample and measuring an increase in uroporphyrin I. In another aspect, the method further comprises the step of measuring uroporphyrinogen III in the sample. In another aspect, the method further comprises the step of measuring PBG in a tissue sample by inhibiting uroporphyrinogen III synthase (UROS) activity. In another aspect, the porphobilinogen deaminase is a bacterial, yeast, or human porphobilinogen deaminase. In another aspect, the porphobilinogen deaminase is immobilized on a substrate. In another aspect, the method further comprises the step of measuring the amount of porphyrin in an unreacted sample, and subtracting the amount of porphyrin in an unreacted sample from the reacted sample. In another aspect, the method further comprises the step of treating the subject having elevated porphobilinogen with carbohydrate loading or hemin, RNA interference, or gene therapy.

In another embodiment, the present invention includes a kit comprising: a vial for collecting a sample; a vial with a porphobilinogen deaminase; a solution for catalyzing a porphobilinogen deaminase reaction with a porphobilinogen in the sample; and instructions for correlating the amount of porphyrin in the sample to an amount of porphobilinogen in the sample, wherein the ratio of porphobilinogen to porphyrin is 4:1, after exposing the porphyrin to a 399 nm wavelength light source. In one aspect, a sample is a urine sample. In another aspect, a sample is from a subject suspected of having acute intermittent porphyria (AIP), hereditary coproporphyria (HCP), or variegate porphyria (VP). In another sample, the porphobilinogen deaminase is a bacterial, yeast, or human porphobilinogen deaminase. In another sample, the porphobilinogen deaminase is immobilized on a substrate. In another sample, the instructions further comprise subtracting the amount of porphyrin in an unreacted sample from the reacted sample.

In another embodiment, the present invention includes a method for treating a patient having elevated porphobilinogen comprising: obtaining a urine sample suspected of having a porphobilinogen; catalyzing a reaction of the urine sample with a porphobilinogen deaminase to form a porphyrin; exposing the porphyrin to a light source having a 399 nm wavelength; detecting fluorometrically the amount of porphyrin in the urine sample; correlating the amount of porphyrin in the urine sample to an amount of porphobilinogen in the sample, wherein the ratio of porphobilinogen to porphyrin is 4:1, wherein the patient is suspected of having at least one of acute intermittent porphyria (AIP), hereditary coproporphyria (HCP), or variegate porphyria (VP); and treating the patient with elevated porphobilinogen with carbohydrate loading or hemin, RNA interference, or gene therapy.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows a linear increase in porphyrin concentration after adding hrPBGD as related to the original porphobilinogen concentration in 3 urine sample from patients with acute intermittent porphyria.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The following terms have the meanings as defined herein.

Porphyrias are metabolic disorders caused by altered activities of enzymes within the heme biosynthetic pathway. Porphyrias can cause neurovisceral manifestations (e.g., abdominal pain, motor and sensory peripheral neuropathy, neuropsychiatric changes) and/or cutaneous photosensitivity (either chronic and blistering or acute and mostly nonblistering).

As used herein, “PBGD” refers to porphobilinogen [PBG] deaminase, also known as hydroxymethylbilane synthase [HMBS]. PBGD is the third enzyme in the heme biosynthetic pathway. Mutations in PBGD/HMBS cause acute intermittent porphyria (AIP).

As used herein, “AIP” refers to Acute intermittent porphyria. AIP is the prototypical and most common acute, neurovisceral porphyria. Cutaneous manifestations do not occur, except, rarely, in association with advanced renal disease. Fecal and plasma porphyrins are normal or modestly elevated. In 9 out of 10 patients with AIP, the level of erythrocyte PBGD activity is approximately half normal.

As used herein, “HCP” refers to Hereditary coproporphyria. HCP produces neurovisceral attacks and, less commonly, blistering cutaneous manifestations. Biochemically, HCP can be differentiated from other acute porphyrias because it produces a markedly increased concentration of coproporphyrin III in urine and especially in feces, with little increase in fecal protoporphyrin.

As used herein, “VP” refers to Variegate porphyria. VP produces neurovisceral attacks; blistering cutaneous manifestations are also common, often leading to an incorrect diagnosis of porphyria cutanea tarda (PCT), a cutaneous porphyria without neurovisceral symptoms or PBG elevation. Biochemically, VP is characterized by elevated plasma porphyrins and a plasma fluorescence peak at approximately 626 nm as well as increased fecal coproporphyrin III and protoporphyrin.

Normal levels of plasma porphyrins are typically <1 mcg/dL (higher in individuals with end-stage renal disease) [25]. Elevations in plasma and urine total porphyrins are found in all active cases with blistering cutaneous porphyrias [(PCT, hepatoerythropoietic porphyria (HEP), VP, HCP, and congenital erythropoietic porphyria (CEP)], with the degree of elevation generally reflecting the severity of the lesions. For example, in mild cases of PCT, the degree of elevation may be small.

A need remains for a reliable, inexpensive, and rapid method for detecting porphyrias. The method takes a new approach, which is to derivative PBG by a highly specific enzyme reaction, and measure that derivative fluorometrically. PBG is the natural substrate for PBG deaminase [also known as hydroxymethylbilane (HMB) synthase (HMBS)], the third enzyme in the heme biosynthetic pathway. The product HMB is a linear tetrapyrrole and is the substrate for the next enzyme in the pathway, uroporphyrinogen III synthase—UROS. HMB is unstable, and in the absence of UROS cyclizes nonenzymatically to form uroporphyrinogen I, which on exposure to light and O₂ will oxidize to uroporphyrin I—an oxidized porphyrin that is fluorescent. This sequence is the basis for assays of PBGD activity in, for example, erythrocytes.

The method of the present invention measures a derivative of PBG fluorometrically. Uroporphyrin I, the PBG derivative, is the end result of an enzymatic reaction beginning with PBG. The inventor has created a method to quantify the amount of PBG in urine by correlating uroporphyrin I levels to PBG levels. PBG is only elevated in the 3 most common acute porphyrias—AIP, HCP and VP.

The method is facilitated by the availability of recombinant human (hrPBGD), which can be prepared in large amounts and used as a reagent for transforming PBG, but not urobilinogen and other interfering substances in urine, to uroporphyrin I. Recombinant PBGD from other species is expected to function just as well and could be used instead of human enzyme, if more readily obtained or prepared. For example, a yeast plasmid can be purchased containing the yeast PBGD gene (known as hemC), which can be cloned in bacteria to produce large amounts of PBGD for laboratory use. The human enzyme was used because of its availability from the Porphyrias Consortium, and because it is quite stable in solution even at ambient temperature. Therefore, it was used in the present studies.

The present inventor used the following method to show that PBGD can be used to generate a fluorescent derivative (uroporphyrin I) to assess the amount of PBG in human urine. The method involves adjusting the pH of urine to an optimal pH of 7.6, adding human PBGD, incubating at 37° C., transforming HMB to uroporphyrin I by light exposure, extracting uroporphyrin I with ethyl acetate/acetic acid and then back extracting it into HCl, followed by fluorometric measurement.

Materials, reagents and instruments: Tris buffer 50 mM, pH 7.6; 10% (NH₄)²CO₃ solution; Human recombinant porphobilinogen deaminase (hrPBGD); 2:1 (v/v) ethyl acetate/acetic acid solution; 0.5 M HCl; Coproporphyrin III standard (Frontier Scientific); Positive displacement micropipette, 1-10 μL; Disposable capillaries/pistons 10/100 μL; Adjustable or fixed volume micropipettes; Borosilicate glass tubes 6×50 mm; Water bath (37° C.); and Spectrofluorometer (Horiba).

Method. (1) Urine sample(s) selected from patients known to have AIP and demonstrated to have high concentrations of PBG. (2) Adjust the pH to 7.6 with 10% (NH4)₂CO3 solution. (3) To 25 μL of urine add 4 μL human recombinant PBGD (hrPBGD) in 50 mM Tris buffer, pH 7.6. (4) A sample blank is prepared, with 25 μL of urine and 4 μL 50 mM Tris buffer, pH 7.6 to correct for endogenous fluorescence. (5) The reaction mixture is incubated at 37° C. for 45 minutes to generate HMB. (6) The reaction is stopped by placing the samples in an ice water bath for 10 minutes and exposed to light to convert HMB to uroporphyrin I. (7) Ethyl acetate-acetic acid (2:1) is added and shaken to extract uroporphyrin I. (8) Uroporphyrin I is back extracted with 0.5M HCl. (9) Fluorescence is recorded at excitation wavelength 399 nm and emission wavelength from 620 to 700 nm. Uroporphyrin I concentration (nmol/L) is calculated for each sample and respective blanks using coproporphyrin III as a standard.

The present inventor measured porphyrins produced by this method using 3 urine samples. A potential problem regarding urine samples from AIP patients is that in addition to PBG, urine porphyrins are also increased. However, the levels of PBG are generally an order of magnitude or more greater than porphyrins. Nevertheless, it was important to demonstrate that a substantial increase in porphyrins was observed after incubation with hrPBGD when urine samples already contained elevated porphyrin levels.

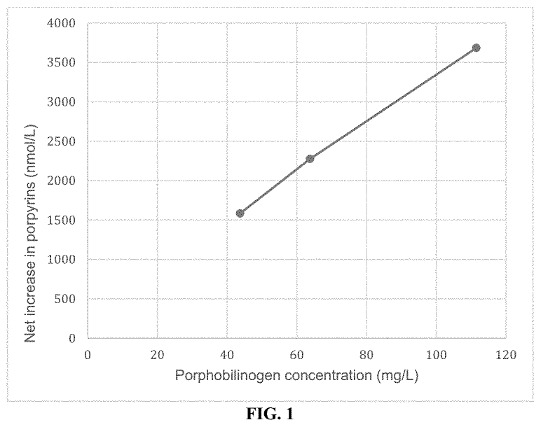

As shown in Table 1, the PBG concentrations in the 3 urine samples from patients with AIP were all very high, i.e. 43.7 to 111.5 (ref 0-7) mg/L, as is typical during attacks of the acute porphyrias. Incubation with hrPBGD led to generation of amounts of porphyrin that exceeded the amounts that were present at baseline (in the sample blank incubated without enzyme). This substantial increase in porphyrins occurred even though the amount of excess PBG converted to porphyrins was only about 3% (Table 1). As shown in FIG. 1, the increase in porphyrins, was linearly related to the original concentration of PBG.

TABLE 1
Formation of porphyrins as measured fluorometrically after incubation of
three urine samples with high concentrations of PBG with hrPBGD.

| | | | | | | | Net |
|---|---|---|---|---|---|---|---|
| | | | | Porphyrins after incubation | | | Porphyrin |
| | PBG in Urine Samples* | | | (nmol/L) | | | Formation |
| | | | Porphyrin | | | Net | (% of |
| Urine | | | equivalence | Without | With | Porphyrin | PBG |
| Samples | (mg/L) | (umol/L) | (nmol/L) | PBGD | PBGD | formation | present) |
| 1 | 43.7 | 193.2 | 48,289 | 943 | 2,525 | 1,583 | 3.3% |
| 2 | 63.8 | 282.0 | 70,499 | 1,568 | 3,844 | 2,276 | 3.2% |
| 3 | 111.5 | 492.8 | 123,208 | 3,294 | 6,978 | 3,684 | 3.0% |

*PBG concentration is expressed as mg/L, umol/L (conversion factor 4.420) and porphyrin equivalence (4 PBG molecules per each porphyrin molecule)

There is currently no method for rapid assessment of the amount in PBG in human urine, and this is extremely important for the timely diagnosis of acute porphyrias. Previous methods have employed solvent extractions or column separations either before or after formation of a chromophore of PBG using Ehrlich reagent. A kit that was suitable for this purpose has been taken off the market, leaving a void in methodology for timely diagnosis of patients with these severe and potentially life threatening conditions. The present invention fills a significant void, and is a method based on new and innovative concepts that can be adapted for the clinical setting.

The method is innovative in that it uses, for the first time, an enzyme to selectively transform PBG to another more readily measurable compound rather than separating it or its chromophore from interfering substances. The enzyme PBGD is highly specific for PBG, which is its natural substrate, and selects PBG and not other substances for transformation to a fluorescent porphyrin—uroporphyrin I—with intermediate formation of HMB and uroporphyrinogen I. While others have measured PBGD activity in erythrocytes and other tissues, it has never been used for measurement of PBG in biological samples such as urine.

The data recorded here demonstrates that hrPBGD can be used to assess PBG in urine, as evidenced by the data showing that the amount of porphyrin generated in the presence of hrPBGD was greater than the amount of porphyrin originally present, and was proportional to the concentration of PBG in the urine samples tested.

Thus, the innovative method taught herein can be used in a clinical setting, especially as a rapid test for elevated urine PBG. The present invention can also be used to develop an instrument or kit that with the needed reagents, including rPBGD, and supplies, and also tools for measuring or assessing visually an increase in fluorescence indicating that a large amount of PBG is found in the tested sample. Separate instrument or kits could be adapted for quantitative or qualitative assessment of urine for elevated PBG.

Finally, based on the present invention it is possible to develop a filter paper test, as is used for diagnosis of a number of other genetic conditions. Human PBGD is remarkably stable in solution, even at ambient temperature, and its stability and reactivity with a substrate can be applied to filter paper. Urine is applied to the enzyme on filter paper, and after incubation, the increase in porphyrin fluorescence can be observed as an indication of a diagnostic increase in PBG or for semiquantitive estimation of the concentration of PBG in the urine sample. If visual assessment is inadequate, instrumental approaches, such as surface fluorescence can be used for rapid measurement of the increase in porphyrins on the filter paper.

Other embodiments of the method are contemplated. For example, greater specificity for PBG is possible if, after adding PBGD to urine or plasma, one measures an increase in uroporphyrin I rather than total uroporphyrin (isomers I and III) or total porphyrins (including coproporphyrins). Greater specificity is possible because HMB, the immediate product of PBGD, nonenzymatically forms uroporphyrinogen I, and is then autooxidized to uroporphyrin I. Therefore, although other porphyrins are increased in plasma and urine in acute porphyrias, only uroporphyrin I should increase after addition of rPBGD to urine or plasma. On the other hand, in tissues such as liver HMB is preferentially transformed to uroporphyrinogen III by the next enzyme in the pathway, uroporphyrinogen III synthase (UROS), which is not found in urine or plasma. If a more specific method such as this were applied to measuring PBG in tissues, there are strategies for inhibiting UROS activity that could be used.

### EXAMPLE 1

A 30-year-old woman presented to a hospital ED with abdominal pain, nausea, vomiting, and diarrhea. The pain required morphine for relief. She was hospitalized for two weeks for a suspected intestinal infection. The evaluation was negative, including a computed tomography (CT) scan and upper and lower endoscopies. She gradually improved and was discharged.

The same symptoms recurred two years later, resulting in multiple ED visits. She reported some increase in alcohol intake to compensate for stressful circumstances. She was admitted to a psychiatric unit with mental status changes and hallucinations and then transferred to an ED for evaluation of abdominal pain. In the ED, she had a grand mal seizure associated with hyponatremia. She was admitted to a medical unit with tachycardia and hypertension (heart rate 120 beats per minute; blood pressure 174/114), along with disorientation; there were no focal neurologic signs.

Examination of the cerebrospinal fluid showed no abnormalities; MRI of the brain showed multiple areas of subcortical signal abnormalities. Electroencephalogram (EEG) was abnormal with recurring single and multiple spike and sharp discharge activity appearing to arise from the left anterior temporal region. Laboratory testing revealed increased aminotransferases (ALT 114 international units/L, AST 94 international units/L), which were attributed to alcohol.

Phenytoin was started for seizures. Abdominal pain and hyponatremia worsened (serum sodium 116 mEq/L). The syndrome of inappropriate ADH (SIADH) was suspected and attributed to fluoxetine. An abnormal hepatobiliary scan led to laparoscopic removal of a histologically normal gallbladder with no gallstones. She was discharged with diagnoses of alcohol withdrawal and alcoholic liver disease and referred for rehabilitation. Urine porphyrins were ordered and reported as “positive” after discharge, but the ordering physician was unable to contact the patient; she had moved to another part of the country.

She had continuing and progressive symptoms, and she was hospitalized after developing muscle weakness. This progressed to quadriparesis and respiratory failure complicated by aspiration pneumonia. Urinary PBG was 44 mg/24 hours (reference range 0 to approximately 4), and a diagnosis of AIP was made. Harmful drugs (including phenytoin) were stopped. She improved gradually with intravenous glucose but was not treated with hemin. Gradual improvement began, and she was discharged for prolonged physical therapy and rehabilitation. Recovery was almost complete, but some objective muscle weakness, painful hyperesthesia of the legs, and impaired short-term memory persisted. She continued to have attacks one to two times yearly, sometimes in the luteal phase of her menstrual cycle.

This case illustrates many of the challenges related to diagnosing and treating AIP, including the delayed recognition of the classic features, the potential benefits of timely diagnosis, and the importance of appropriate treatment with hemin for severe symptoms. Recovery can be complete or nearly complete with treatment, but lasting damage may persist. Her diagnosis could have been made before discharge if a rapid and reliable testing method has been available at the treating hospital.

Initial testing (acute hepatic porphyria suspected)—Importantly, the acute hepatic porphyrias are readily ruled in or out at the time of symptoms by a simple urine test for porphobilinogen (PBG), which is both highly sensitive and highly specific, is available. Thus, urine PBG is the most important first-line screening test when acute porphyria is suspected.

A timed urine collection is not needed; a spot urine sample is sufficient and generally preferred. Collecting urine for 24 hours can cause unnecessary delays in diagnosis. Highly dilute urine can give falsely negative results; thus, if an initial test result is expressed as PBG per liter of urine, the urine creatinine should also be measured on the same sample and the PBG result expressed per gram (or millimol) of urine creatinine. However, a very high result expressed per liter is diagnostically meaningful.

If results are positive (e.g., PBG level>10 mg per g creatinine [or >10 mg/L]), treatment with hemin can (and typically should) be initiated without delay if clinical manifestations are severe.

If results are negative (e.g., PBG level<5 mg per g creatinine [or <5 mg/L]), testing for other conditions in the differential diagnosis is appropriate.

PBG can also be measured in serum or plasma, which is essential if acute porphyria is suspected in a patient with advanced renal disease. However, in patients with acute porphyria and normal kidney function, PBG concentrations are higher in urine, so urinary measurements are more sensitive. The proposed method can be adapted testing for elevated PBG in serum or plasma.

Normal urinary excretion of PBG is <2 to 4 mg (<9 to 18 micromol) per day. Amounts expressed per gram creatinine are roughly the same, since adults excrete 0.7 to 2.0 grams of creatinine daily. PBG excretion during an acute porphyria attack is markedly elevated, with typical values at least 5 to 10 times the upper limit of normal (e.g., >10 to 100 mg/day [or per g/creatinine]; >44 to >440 micromol/day).

Additional nuances of different methods for PBG testing (mass spectrometry versus ion exchange chromatography) are discussed above.

A rapid test kit for urinary PBG is no longer available, so samples at most sites must be sent to a referral laboratory. When a result is needed urgently at present, the laboratory should be contacted and asked to expedite the testing. The method can be adapted for use at local medical centers as well by centralized laboratories.

The most common acute hepatic porphyria is AIP. Less common possibilities include hereditary coproporphyria (HCP), variegate porphyria (VP), and delta-aminolevulinic acid (ALA) dehydratase (ALAD) porphyria (ADP; which is very rare). To help distinguish among these, total porphyrins are also measured at the time of presentation.

Total porphyrins are elevated in all four acute porphyrias. In HCP and VP, total porphyrins may remain elevated for a longer period of time than PBG. However, urine porphyrin elevations are very nonspecific and occur in most other porphyrias and in many medical conditions other than porphyrias.

ALA can also be measured but is not essential at screening. Normal urinary excretion of ALA is <7 mg (<53 micromol) per day. In an acute attack of AIP, HCP, or VP, urinary ALA excretion is elevated, but generally less so than PBG (when expressed in mg).

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

U.S. Pat. No. 6,537,777.

Takeshi Uchida, Takumi Funamizu, Minghao Chen, Yoshikazu Tanaka, and Koichiro Ishimori, Heme Binding to Porphobilinogen Deaminase from Vibrio cholera Decelerates the Formation of 1 Hydroxymethylbilane, ACS Chem. Biol. 2018, 13, 750-760.

What is claimed is:

1. A method for detecting porphobilinogen fluorometrically comprising:
obtaining a sample suspected of having a porphobilinogen;
catalyzing a reaction in the sample with a porphobilinogen deaminase to form a porphyrin reaction sample;
exposing the porphyrin to a light source having a 399 nm wavelength;
detecting fluorometrically the amount of porphyrin in the sample at 620 to 700 nm; and
correlating the amount of porphyrin in the reaction sample to an amount of porphobilinogen in the sample, wherein the ratio of porphobilinogen to porphyrin is 4:1.

2. The method of claim 1, wherein the sample is a urine sample.

3. The method of claim 1, wherein the sample is from a subject suspected of having at least one of: acute intermittent porphyria (AlP), hereditary coproporphyria (HCP), or variegate porphyria (VP).

4. The method of claim 1, further comprising the step of adding porphobilinogen deaminase (PBGD) to the sample and measuring an increase in uroporphyrin I.

5. The method of claim 1, further comprising the step of measuring uroporphyrinogen III in the sample.

6. The method of claim 1, further comprising the step of measuring PBG in a tissue sample by inhibiting uroporphyrinogen III synthase (UROS) activity.

7. The method of claim 1, wherein the porphobilinogen deaminase is a human porphobilinogen deaminase.

8. The method of claim 1, wherein the porphobilinogen deaminase is immobilized on a substrate.

9. The method of claim 1, further comprising the step of measuring the amount of porphyrin in an unreacted sample, and subtracting the amount of porphyrin in an unreacted sample from the reacted sample.

10. The method of claim 1, wherein the porphobilinogen deaminase is a bacterial, yeast, or human porphobilinogen deaminase.

11. The method of claim 1, further comprising the step of treating the subject having elevated porphobilinogen with carbohydrate loading or hemin, RNA interference, or gene therapy.

12. A method for treating a patient having elevated porphobilinogen comprising:
obtaining a urine sample suspected of having a porphobilinogen;
catalyzing a reaction of the urine sample with a porphobilinogen deaminase to form a porphyrin;
exposing the porphyrin to a light source having a 399 nm wavelength;
detecting fluorometrically the amount of porphyrin in the urine sample;
correlating the amount of porphyrin in the urine sample to an amount of porphobilinogen in the sample, wherein the ratio of porphobilinogen to porphyrin is 4:1, wherein the patient is suspected of having at least one of acute intermittent porphyria (AIP), hereditary coproporphyria (HCP), or variegate porphyria (VP); and
treating the patient with elevated porphobilinogen with carbohydrate loading or hemin, RNA interference, or gene therapy.

13. A kit comprising:
a vial for collecting a sample;
a vial with a porphobilinogen deaminase;
a solution for catalyzing a porphobilinogen deaminase reaction with a porphobilinogen in the sample; and
instructions for correlating the amount of porphyrin in the sample to an amount of porphobilinogen in the sample, wherein the ratio of porphobilinogen to porphyrin is4:1, after exposing the porphyrin to a 399 nm wavelength light source.

14. The kit of claim 13, wherein a sample is a urine sample.

15. The kit of claim 13, wherein a sample is from a subject suspected of having acute intermittent porphyria (AIP), hereditary coproporphyria (HCP), or variegate porphyria (VP).

16. The kit of claim 13, wherein the porphobilinogen deaminase is a bacterial, yeast, or human porphobilinogen deaminase.

17. The kit of claim 13, wherein the porphobilinogen deaminase is immobilized on a substrate.

18. The kit of claim 13, wherein the instructions further comprise subtracting the amount of porphyrin in an unreacted sample from the reacted sample.
